(19) United States
(12) Patent Application Publication
(10) Pub. No.: US 2022/0041649 A1
(43) Pub. Date: Feb. 10, 2022

(54) Methods for Synthesis of Radionuclide Complex

(71) Applicant: Advanced Accelerator Applications (Italy) S.R.L., Pozzilli (IT)

(72) Inventors: Lorenza Fugazza, Ivrea (IT); Francesco De Palo, Ivrea (IT); Donato Barbato, Ivrea (IT); Maurizio F. Mariani, Ivrea (IT); Giovanni Tesoriere, Colleretto Giacosa (IT); Clementina Brambati, Torino (IT)

(73) Assignee: Advanced Accelerator Applications (Italy) S.R.L., Pozzilli (IT)

(21) Appl. No.: 17/331,927

(22) Filed: May 27, 2021

Related U.S. Application Data
(63) Continuation of application No. 16/393,103, filed on Apr. 24, 2019, now abandoned.
Application No. 16/393,103 is a continuation of application No. PCT/EP2018/079909, filed on Oct. 31, 2018.

(51) Int. Cl. C07K 1/13; A61K 51/04; A61K 51/08; A61K 51/12; C07K 14/655; A61K 9/08

(52) U.S. Cl. CPC C07K 1/13; A61K 51/0482; A61K 51/083; A61K 51/088; C07K 14/655; A61K 9/08; A61K 51/121

(57) ABSTRACT

The present disclosure relates to the synthesis of radionuclide complex solutions, in particular for their use in the commercial production of radioactive drug substances, for diagnostic and/or therapeutic purposes. In particular, the synthesis method comprises the following steps in the following order:
a. providing a radionuclide precursor solution into a first vial,
b. transferring the radionuclide precursor solution into a reactor,
c. providing a reaction buffer solution into said first vial containing residual radionuclide precursor solution,
d. transferring the buffer reaction solution and residual radionuclide precursor solution from said first vial into the reactor,
e. transferring a peptide solution comprising the somatostatin receptor binding peptide linked to a chelating agent, into the reactor,
f. reacting the somatostatin receptor binding peptide linked to a chelating agent with said radionuclide in the reactor to obtain the radionuclide complex,
g. recovering said radionuclide complex.

### TECHNICAL FIELD

The present disclosure relates to the synthesis of radionuclide complex solutions, in particular for their use in the commercial production of radioactive drug substances, for diagnostic and/or therapeutic purposes.

### BACKGROUND ART

The concept of targeted drug delivery is based on cell receptors which are overexpressed in the target cell in contrast to the not-to-be-targeted cells. If a drug has a binding site to those overexpressed cell receptors it allows the delivery of the drug after its systemic administration in high concentration to those target cells while leaving other cells, which are not of interest, unaffected. For example, if tumor cells are characterized by an overexpression of a specific cell receptor, a drug with binding affinity to said receptor will accumulate in high concentration in the tumor tissue after intravenous infusion while leaving the normal tissue unaffected.

This targeted drug delivery concept has also been used in radiomedicine to selectively deliver radionuclides to the target cells for diagnostic or therapeutic purposes. For this radiomedicinal application, the target cell receptor binding moiety is typically linked to a chelating agent which is able to form a strong complex with the metal ions of a radionuclide. This radionuclide complex is then delivered to the target cell and the decay of the radionuclide is then releasing high energy electrons, positrons or alpha particles as well as gamma rays at the target site.

Such radioactive drug substance is preferably produced in a shielded closed-system; manufacturing, purification and formulation process of the drug substance being part of a continuous process. Indeed, the decay of the radionuclide does not allow enough time for any interruption. Therefore, no tests may preferably be performed at critical steps and no synthesis intermediate may be isolated and controlled in the course of production.

Thus, it is desirable to provide automated synthesis methods for the production of such radionuclide complex. Ideally, an automated synthesis method for the production of radionuclide complex as radioactive drug substance may have also the following advantages:
A high labeling yield correlating with high radiochemical purity,
A high labeling yield with minimized level of free (uncomplexed) radionuclide,
A production of a large number of doses par batch.

### SUMMARY OF THE DISCLOSURE

The present disclosure relates to a method for the synthesis of a radionuclide complex formed by a radionuclide and a somatostatin receptor binding peptide linked to a chelating agent characterized in that said method comprises the following steps in the following order:
a) providing a radionuclide precursor solution into a first vial,
b) transferring the radionuclide precursor solution into a reactor,
c) providing a reaction buffer solution into said first vial containing residual radionuclide precursor solution,
d) transferring the reaction buffer solution and residual radionuclide precursor solution from said first vial into the reactor,
e) transferring a solution comprising the somatostatin receptor binding peptide linked to a chelating agent, into the reactor,
f) reacting the somatostatin receptor binding peptide linked to a chelating agent with said radionuclide in the reactor to obtain the radionuclide complex, and,
g) recovering said radionuclide complex.

The present disclosure also relates to an aqueous pharmaceutical solution comprising a radionuclide complex, which solution is obtainable or directly obtained by the method as described herein.

### BRIEF DESCRIPTION OF THE DRAWINGS

FIGS. 1 and 2 shows the main steps of the manufacturing process as described in the Examples.

FIGS. 3A and 3B show the layout of the cassette for use in the manufacturing process before and after modification.

FIG. 4A: Final cassette installation for use in the TRACERlab MX synthesis module.

FIG. 4B: Final cassette installation for use in the Trasis synthesis module.

### DETAILED DESCRIPTION

The present disclosure relates to the synthesis of a radionuclide complex formed by a radionuclide and a somatostatin receptor binding peptide linked to a chelating agent; said method comprises:
a) providing a radionuclide precursor,
b) providing a somatostatin receptor binding peptide linked to a chelating agent,
c) providing a reaction buffer solution,
d) mixing said radionuclide precursor and said somatostatin receptor binding peptide linked to a chelating agent with the buffer reaction solution in a reactor,
e) reacting the somatostatin receptor binding peptide linked to a chelating agent with said radionuclide in the reactor to obtain the radionuclide complex,
f) recovering said radionuclide complex.

Such radionuclide complex is preferably a radioactive drug substance for use in nuclear medicine as diagnostic or therapeutic agent.

The methods of the present disclosure are advantageously amenable to automation. Accordingly, in preferred embodiments, the methods of the present disclosure are automated synthesis methods. The term “automated synthesis” refers to a chemical synthesis that is performed without human intervention. Advantageously, the synthesis according to the method of the disclosure may provide a production of radionuclide complex drug substance with specific activity superior to 45 GBq in a final batch volume which is comprised between 13 and 24 mL, i.e. a specific activity concentration higher than 1875 MBq/mL, for example between 1875 and 3500 MBq/mL. For example, considering that a single dose of ¹⁷⁷Lu-DOTATOC or ¹⁷⁷Lu-DOTATATE would typically be comprised between 4 and 5 GBq (e.g. about 4.7 GBq), the present method may provide mother solution of a concentrate of radionuclide complex (e.g. ¹⁷⁷Lu-DOTATOC or ¹⁷⁷Lu-DOTATATE) for obtaining at least 5, preferably at least 6, 7, 8, 9, 10 or more individual doses of the drug product after dilution and formulation of said mother solution.

The synthesis methods may also advantageously provide a synthesis yield superior to 60%.

Definitions

As used herein, the term “radionuclide precursor solution” refers to the solution containing the radionuclide for use as a starting material. The methods of the present disclosure are particularly adapted for use of radionuclide of metallic nature and which are useful in medicine for diagnostic and/or therapeutic purposes. Such radionuclide includes, without limitation, the radioactive isotopes of In, Tc, Ga, Cu, Zr, Y and Lu, and in particular: ¹¹¹In, ^{99m}Tc, ⁶⁸Ga, ⁶⁴Cu, ⁸⁹Zr, ⁹⁰Y, ¹⁷⁷Lu. The metallic ions of such radioisotopes are able to form non-covalent bond with the functional groups of the chelating agent, e.g. amines or carborboxylic acids.

In a preferred embodiment, the radionuclide precursor solution comprises lutetium-177 (¹⁷⁷Lu). For example, the radionuclide precursor solution comprises ¹⁷⁷LuCl₃ in HCl solution. In one specific embodiment, the radionuclide precursor solution is a ¹⁷⁷LuCl₃ in HCl solution with specific activity concentration higher than 40 GBq/mL.

Typically, a ¹⁷⁷Lu chloride solution for one batch for synthesis of ¹⁷⁷Lu-DOTATOC or ¹⁷⁷Lu-DOTATATE mother solution may have specific activity of 74 GBq or 148 GBq (±20%).

As used herein, the term “somatostatin receptor binding peptide” refers to a peptidic moiety with specific binding affinity to somatostatin receptor. Such somatostatin receptor binding peptide may be selected from octreotide, octreotate, lanreotide, vapreotide, and pasireotide, preferably selected from octreotide and octreotate.

As used herein, the term “chelating agent” refers to an organic moiety comprising functional groups that are able to form non-covalent bonds with the radionuclide at the reacting step of the method and, thereby, form stable radionuclide complex. The chelating agent in the context of the present invention may be 1,4,7,10-Tetraazacyclododecane-1,4,7,10-tetraacetic acid (DOTA), diethylentriaminepentaacetic acid (DTPA), nitrilotriacetic acid (NTA), ethylenediaminetetraacetic acid (EDTA), 1,4,7,10-tetraazacyclododecane-1,4,7-triacetic acid (DO3A), 1,4,7-triazacyclononane-1,4,7-triacetic acid (NOTA), or mixtures thereof, preferably is DOTA.

Such chelating agent is either directly linked to the somatostatin receptor binding peptide or connected via a linker molecule, preferably it is directly linked. The linking bond(s) is (are) either covalent or non-covalent bond(s) between the cell receptor binding organic moiety (and the linker) and the chelating agent, preferably the bond(s) is (are) covalent.

According to preferred embodiments of the synthesis method of the present disclosure, the somatostatin receptor binding peptide linked to the chelating agent is selected from DOTA-OC, DOTA-TOC (edotreotide), DOTA-NOC, DOTA-TATE (oxodotreotide), DOTA-LAN, and DOTA-VAP, preferably selected from DOTA-TOC and DOTA-TATE, more preferably DOTA-TATE.

Particularly preferred embodiments encompass synthesis methods of ¹⁷⁷Lu-DOTA-TOC (¹⁷⁷Lu-edotreotide) or ¹⁷⁷Lu-DOTA-TATE (¹⁷⁷Lu-oxodotreotide), preferably ¹⁷⁷Lu-DOTA-TATE (¹⁷⁷Lu-oxodotreotide). In such embodiments for the synthesis of ¹⁷⁷Lu-DOTA-TOC (¹⁷⁷Lu-edotreotide) or ¹⁷⁷Lu-DOTA-TATE (¹⁷⁷Lu-oxodotreotide), the radionuclide precursor solution comprises ¹⁷⁷Lu in HCl solution, and the peptide solution comprises DOTA-TOC or DOTA-TATE respectively.

For example, DOTA-TATE or DOTA-TOC peptide solution is an aqueous solution comprising between 0.8 mg/mL and 1.2 mg/mL of DOTA-TATE or DOTA-TOC, e.g. lmg/mL. The peptide solution may be obtained by dissolution of a dry powder of the peptide salt in sterile water, prior to starting the synthesis method. Typically, a peptide solution for one batch may contain 2 or 4 mg (±5%) of DOTA-TATE or DOTA-TOC.

As used herein, the reaction buffer solution is an aqueous solution preferably comprising at least a stabilizer against radiolytic degradation and a buffer for a pH from 4.0 to 6.0, preferably from 4.5 to 5.5.

As used herein, the term “stabilizer against radiolytic degradation” refers to a stabilizing agent which protects organic molecules against radiolytic degradation, e.g. when a gamma ray emitted from the radionuclide is cleaving a bond between the atoms of an organic molecules and radicals are forms, those radicals are then scavenged by the stabilzer which avoids the radicals undergo any other chemical reactions which might lead to undesired, potentially ineffective or even toxic molecules. Therefore, those stabilizers are also referred to as “free radical scavengers” or in short “radical scavengers”. Other alternative terms for those stabilizers are “radiation stability enhancers”, “radiolytic stabilizers”, or simply “quenchers”.

Stabilizer(s) present in the reaction buffer solution may be selected from gentisic acid (2,5-dihydroxybenzoic acid) or salts thereof, ascorbic acid (L-ascorbic acid, vitamin C) or salts thereof (e.g. sodium ascoorbate), methionine, histidine, melatonine, ethanol, and Se-methionine, preferably selected from gentisic acid or salts thereof. In specific embodiments, the reaction buffer solution does not include ascorbic acid, preferably it includes gentisic acid as stabilizer agent but not ascorbic acid.

A “buffer for a pH from 4.0 to 6.0, preferably from 4.5 to 5.5” may be an acetate buffer, citrate buffer (e.g. citrate+HCl or citric acid+Disodium hydrogenphosphate) or phosphate buffer (e.g. Sodium dihydrogenphosphate+Disodium hydrogenphosphate), preferably said buffer is an acetate buffer, preferably said acetate buffer is composed of acetic acid and sodium acetate.

For example, a reaction buffer solution is an aqueous solution comprising between 35 and 45 mg/mL of gentisic acid, e.g. 39 mg/mL of gentisic acid, in an acetate buffer. The reaction buffer solution may be obtained by dissolution of a dry powder (lyophililsate) of gentisic acid in acetate buffer in sterile water, prior to starting the synthesis method. Typically, a reaction buffer solution for one batch synthesis of a mother solution of ¹⁷⁷Lu-DOTA-TOC (¹⁷⁷Lu-edotreotide) or ¹⁷⁷Lu-DOTA-TATE (¹⁷⁷Lu-oxodotreotide) may contain 157 mg or 314 mg (+5%) of gentisic acid as the sole stabilizing agent.

The Mixing and Reacting Steps of the Synthesis Method

The synthesis of the radionuclide complex starts after the mixing of three solutions in a reactor vial:
the radionuclide precursor solution, e.g., the Lu-177 chloride solution,
the reaction buffer solution, e.g. a solution comprising gentisic acid,
the peptide solution, e.g. a solution comprising DOTA-TOC or DOTA-TATE, preferably DOTA-TATE.

According to a preferred embodiment of the synthesis method, the above three solutions are transferred into the reactor vial in the following order:

1) the radionuclide precursor solution, e.g., the Lu-177 chloride solution,
2) the reaction buffer solution, e.g. a solution comprising gentisic acid, and,
3) the peptide solution, e.g. a solution comprising DOTA-TOC or DOTA-TATE, preferably DOTA-TATE.

In particular, according to an advantageous aspect of such preferred embodiment, the reaction buffer solution is mixed with the radionuclide precursor solution prior to its mixing with the peptide solution.

More specifically, the inventors have noticed that incomplete transfer of high concentrated radionuclide precursor solution have a substantial impact in the labeling yield, and therefore the synthesis yield. Accordingly, in a more preferred embodiment, said synthesis method comprises the following steps in the following order:
a. providing a radionuclide precursor solution into a first vial,
b. transferring the radionuclide precursor solution into a reactor,
c. providing a reaction buffer solution into said first vial containing residual radionuclide precursor solution,
d. transferring the buffer reaction solution and residual radionuclide precursor solution from said first vial into the reactor,
e. transferring a peptide solution comprising the somatostatin receptor binding peptide linked to a chelating agent, into the reactor,
f. reacting the somatostatin receptor binding peptide linked to a chelating agent with said radionuclide in the reactor to obtain the radionuclide complex,
g. recovering said radionuclide complex.

According to the above protocol, the buffer reaction solution is advantageously used to rinse the vial containing the radionuclide precursor solution and ensure complete (or almost complete) transfer of radionuclide precursor solution in the reactor, while maintaining relatively high specific activity concentration at labeling time. Typically, in a specific embodiment for the synthesis of ¹⁷⁷Lu-DOTA-TOC (¹⁷⁷Lu-edotreotide) or ¹⁷⁷Lu-DOTA-TATE (¹⁷⁷Lu-oxodotreotide), said radionuclide precursor solution is a ¹⁷⁷LuCl₃ chloride solution, wherein the specific activity at reacting time is at least 370 GBq/mg, preferably between 370 GBq/mg and 1110 GBq/mg.

The reacting step of the synthesis method consists of the chelating of the radionuclide, e.g. Lutetium-177, with the chelating agent (e.g. DOTA for DOTA-TOC or DOTA-TATE). The inventors have also shown that a molar excess of the peptide with respect to the radionuclide is preferable to ensure acceptable radiochemical labelling yields. Accordingly, in another specific embodiment, the molar ratio between the somatostatin receptor binding peptide linked to a chelating agent, e.g., DOTA-TOC or DOTA-TATE, and the radionuclide, e.g. Lutetium-177, at the reacting step is at least 1.2, preferably between 1.5 and 3.5.

Advantageously, in certain preferred embodiments of the synthesis method of the present disclosure, the synthesis method does not comprise any purification step to remove free (non-chelated) Lutetium-177, such as a tC18 solid phase extraction (SPE) purification step. The use of a tC18 cartridge to perform a solid phase extraction (SPE) purification step to remove free (non-chelated) Lutetium-177 presents some disadvantages. In particular, the use of this cartridge may require the elution of the product with ethanol, which is undesired (A. Mathur et al., Cancer Biother. Radiopharm. 2017, 32, 266-273). The use of a tC18 cartridge may also remove the stabilizers, which then need to be added again (S. Maus et al. Int. J. Diagnostic imagin 2014, 1, 5-12).

In certain embodiments, especially for the synthesis of ¹⁷⁷Lu-DOTA-TOC (¹⁷⁷Lu-edotreotide) or ¹⁷⁷Lu-DOTA-TATE (¹⁷⁷Lu-oxodotreotide), the reacting step may be advantageously performed at a pH comprised between 4.5 and 5.5.

In specific embodiments, the reaction time at the reacting step is between 2 and 15 minutes, typically 5 or 12 minutes, and/or the temperature is comprised between 80-100° C., preferably between 90-95° C.

The method may further comprise at least one or more rinsing steps for best recovery of the radionuclide complex formed during the reacting step. Typically, one or more volume of water is added to the reactor and recovered in the final volume comprising the radionuclide complex.

Preferably, the mixture volume at reacting step is between 4 and 12 mL and the final volume containing the radionuclide complex after recovering step (therefore including volume(s) of water for the rinsing steps) is comprised between 13 and 24 mL.

### Specific Embodiments for the Synthesis of ¹⁷⁷Lu-DOTA-TATE (¹⁷⁷Lu-Oxodotreotide) Mother Solution

The synthesis method of the present disclosure may be advantageously used for the synthesis of ¹⁷⁷Lu-DOTA-TATE (¹⁷⁷Lu-oxodotreotide), especially for use as a mother solution for the production of infusion solution of ¹⁷⁷Lu-DOTA-TATE ready-to-use.

As used herein, the term “mother solution” refers to a solution which is used to prepare a final drug product, by dilution in a formulation buffer. The mother solution advantageously enables the preparation of at least 5 therapeutic doses of ¹⁷⁷Lu-DOTA-TATE. For example, a therapeutic dose of ¹⁷⁷Lu-DOTA-TATE for the treatment of somatostatin receptor positive gastroenteropancreatic neuroendocrine tumors comprises a total radioactivity of 7,400 MBq at the date and time of infusion, typically within a final adjusted volume between 20.5 mL and 25.0 mL.

In a specific embodiment for the synthesis of a mother solution of ¹⁷⁷Lu-DOTA-TATE, said synthesis method comprises the following steps in the following order:
a. providing a radionuclide precursor solution into a first vial,
b. transferring the radionuclide precursor solution into a reactor,
c. providing a reaction buffer solution into said first vial containing residual radionuclide precursor solution,
d. transferring the buffer reaction solution and residual radionuclide precursor solution from said first vial into the reactor,
e. transferring a peptide solution comprising the somatostatin receptor binding peptide linked to a chelating agent, into the reactor,
f. reacting the somatostatin receptor binding peptide linked to a chelating agent with said radionuclide in the reactor to obtain the radionuclide complex,
g. recovering said radionuclide complex.
   and the following solutions are used:
(i) said radionuclide precursor solution is a ¹⁷⁷LuCl₃ solution at 74 GBq±20% in a 1-2 mL volume, typically, 1.5 mL,
(ii) said solution comprising the somatostatin receptor binding peptide linked to a chelating agent is a solution comprising 2 mg±5% of DOTA-TATE in a volume comprised between 1.5 and 2.5 mL, typically 2 mL,
(iii) said reaction buffer solution comprises 157 mg of gentisic acid±5% in a volume comprised between 1.5 and 2.5 mL, typically 2 mL, and the pH of the reacting step is comprised between 4.5 and 5.5.

Advantageously, according to the above method, the radionuclide complex recovered at step g may be an aqueous concentrate mother solution comprising ¹⁷⁷Lu-DOTA-TATE at a specific activity at least equal to 45.0 GBq in a final volume between 13 and 24 mL.

In another specific embodiment of the synthesis of a mother solution of¹⁷⁷Lu-DOTA-TATE, said synthesis method comprises the following steps in the following order:
a. providing a radionuclide precursor solution into a first vial,
b. transferring the radionuclide precursor solution into a reactor,
c. providing a reaction buffer solution into said first vial containing residual radionuclide precursor solution,
d. transferring the buffer reaction solution and residual radionuclide precursor solution from said first vial into the reactor,
e. transferring a peptide solution comprising the somatostatin receptor binding peptide linked to a chelating agent, into the reactor,
f. reacting the somatostatin receptor binding peptide linked to a chelating agent with said radionuclide in the reactor to obtain the radionuclide complex,
g. recovering said radionuclide complex.
   and the following solutions are used:
(i) said radionuclide precursor solution is a ¹⁷⁷LuCl₃ at 148 GBq±20% in a 2-3 mL volume, typically, 2.5 mL,
(ii) said solution comprising the somatostatin receptor binding peptide linked to a chelating agent is a solution comprising 4 mg±5% of DOTA-TATE in a volume comprised between 3.5 and 4.5 mL, typically 4 mL,
(iii) said reaction buffer solution comprises 314 mg of gentisic acid±5% in a volume comprised between 3.5 and 5.5 mL, typically 4 mL, and the pH of the reacting step is comprised between 4.5 and 5.5.

Advantageously, according to the above method, the radionuclide complex recovered at step g may be an aqueous concentrate mother solution comprising ¹⁷⁷Lu-DOTA-TATE at a specific activity at least equal to 59.0 GBq, in a final volume between 19 and 24 mL.

The above specific methods enable a synthesis yield that may be higher than 60%.

Synthesis Module with Single Use Kit Cassette

The above described synthesis method may be advantageously automated and implemented in a synthesis module with a single use kit cassette.

For example, a single use kit cassette is installed on the front of the synthesis module which contains the fluid pathway (tubing), reactor vial and sealed reagent vials. The disposable cassette components are made out of materials specifically chosen to be compatible with the reagents used in the process. In particular, the components are designed to minimize potential leaching from surfaces in contact with the fluids of the process while maintaining mechanical performance and integrity of the cassette.

Preferably, the synthesis method is fully automated and the synthesis takes place within a computer assisted system.

A typical kit cassette may include
(1) a reaction vial (reactor),
(2) connections for incoming and outgoing fluids,
(3) spikes for connecting reagent vials, and,
(4) optionally, solid phase cartridges.

The skilled person may adapt commercially available kit cassettes used for the preparation of radiopharmaceuticals such as F-18 Labeled radiopharmaceuticals.

In specific embodiments, the synthesis module and kit cassette comprises the following:
(i) at a first position, a needle is placed for inserting to the top of said first vial containing the radioactive precursor solution,
(ii) at a second position, a needle is placed for inserting to the top of a vial containing said solution comprising the somatostatin receptor binding peptide linked to a chelating agent,
(iii) at a third position, a bag with water for injection is installed, for rinsing steps,
(iv) at a fourth position, the reaction buffer solution is installed, and,
(v) at a fifth position, an extension cable is installed to transfer the radionuclide complex from the synthesis module into a dispensing isolator.

Specific examples of synthesis module and kit cassette are described in the Examples.

The present disclosure also relates to the kit cassette for carrying out the method as defined above, comprising:
(i) a first vessel containing the buffer reaction solution or a lyophilisate of said buffer reaction solution,
(ii) a second vessel containing the peptide solution comprising said somatostatin receptor binding peptide linked to a chelating agent, preferably DOTA-TATE or DOTA-TOC, or a lyophilisate of peptide solution, and,
(iii) a third vessel containing said radionuclide precursor solution, preferably Lutetium-177 chloride solution.

Manufacturing of the Radionuclide Complex as a Drug Product

The skilled person will be able to prepare the radionuclide complex as a drug product using the above described synthesis method.

In specific embodiments of the synthesis method, the synthesis method further comprises a step of diluting the radionuclide complex as recovered from the above synthesis method (typically as a concentrated mother solution) in a formulation buffer.

As used herein, the wording “formulation buffer” refers to the solution that is used to obtain a pharmaceutical aqueous solution which is “ready-to-use”. For example, a formulation buffer of ¹⁷⁷Lu-DOTA-TATE or ¹⁷⁷Lu-DOTA-TOC is an aqueous solution that is used to obtain a solution for infusion of ¹⁷⁷Lu-DOTA-TATE or ¹⁷⁷Lu-DOTA-TOC, preferably at specific activity concentration of 370 MBq/mL (±5%). The formulation buffer may comprise one or more of the following excipients selected from: a sequestering agent (e.g. diethylene triamine pentaacetic acid=pentetic acid=DTPA), a radiolytic stabilizer (e.g. ascorbic acid), and a pH adjuster (e.g. NaOH).

Aqueous Pharmaceutical Solution as Obtained by the Synthesis Methods

The present disclosure also relates to the aqueous pharmaceutical solution obtainable or obtained by the above described synthesis methods of the present disclosure.

In specific embodiments, such aqueous pharmaceutical solution obtainable or obtained by the above described synthesis methods is a mother solution of ¹⁷⁷Lu-DOTA-TATE or ¹⁷⁷Lu-DOTA-TOC, preferably at a specific activity concentration higher than 1875 MBq/mL, typically between 1875 and 3400 MBq/mL.

In other embodiments, further comprising a formulation step, for example as described in the previous paragraph, such aqueous pharmaceutical solution obtainable or obtained by the above described synthesis methods is a solution for infusion of ¹⁷⁷Lu-DOTA-TATE or ¹⁷⁷Lu-DOTA-TOC preferably at specific activity concentration of 370 MBq/mL (±5%).

### Embodiments

1. A method for the synthesis of a radionuclide complex formed by a radionuclide and a somatostatin receptor binding peptide linked to a chelating agent characterized in that said method comprises the following steps in the following order:
   a) providing a radionuclide precursor solution into a first vial,
   b) transferring the radionuclide precursor solution into a reactor,
   c) providing a reaction buffer solution into said first vial containing residual radionuclide precursor solution,
   d) transferring the reaction buffer solution and residual radionuclide precursor solution from said first vial into the reactor,
   e) transferring a solution comprising the somatostatin receptor binding peptide linked to a chelating agent, into the reactor,
   f) reacting the somatostatin receptor binding peptide linked to a chelating agent with said radionuclide in the reactor to obtain the radionuclide complex,
   g) recovering said radionuclide complex.
2. The method of Embodiment 1, wherein said chelating agent is selected from DOTA, DTPA, NTA, EDTA, DO3A, NOC and NOTA, preferably is DOTA.
3. The method of Embodiment 1 or 2, wherein said somatostatin receptor binding peptide is selected from octreotide, octreotate, lanreotide, vapreotide, and pasireotide, preferably selected from octreotide and octreotate.
4. The method of any one of Embodiments 1-3, wherein the somatostatin receptor binding peptide linked to the chelating agent is selected from DOTA-OC, DOTA-TOC (edotreotide), DOTA-NOC, DOTA-TATE (oxodotreotide), DOTA-LAN, and DOTA-VAP, preferably selected from DOTA-TOC and DOTA-TATE, more preferably DOTA-TATE.
5. The method of any one of Embodiments 1-4, wherein said radionuclide complex is ¹⁷⁷Lu-DOTA-TOC (¹⁷⁷Lu-edotreotide) or ¹⁷⁷Lu-DOTA-TATE (¹⁷⁷Lu-oxodotreotide), preferably ¹⁷⁷Lu-DOTA-TATE (¹⁷⁷Lu-oxodotreotide).
6. The method of Embodiment 5, wherein said radionuclide precursor solution is a ¹⁷⁷LuCl₃ chloride solution, wherein the specific activity at reacting step is at least 407 GBq/mg, preferably between 407 GBq/mg and 1110 GBq/mg.
7. The method of any one of Embodiments 1-6, wherein the molar ratio between the somatostatin receptor binding peptide linked to a chelating agent and the radionuclide at the reacting step f) is at least 1.2, preferably between 1.5 and 3.5.
8. The method of any one of Embodiments 1-7, wherein said reaction buffer solution comprises at least a stabilizer against radiolytic degradation, preferably selected from gentisic acid.
9. The method of any one of Embodiments 1-8, wherein said reaction buffer solution comprises sodium acetate.
10. The method of any one of Embodiments 1-9, wherein the reacting step f is performed at a pH comprised between 4.5 and 5.5.
11. The method of any one of Embodiments 1-10, wherein said reaction buffer solution does not contain ascorbic acid.
12. The method of any one of Embodiments 1-11, wherein the reaction time at the labeling step f is between 2 and 15 minutes, typically 5 or 12 minutes, and the temperature is comprised between 80-100° C., preferably between 90-95° C.
13. The method of any one of Embodiments 1-12, further comprising at least one or more rinsing steps for efficient recovery of the radionuclide complex.
14. The method of any one of Embodiments 1-13, wherein the mixture volume at reacting step is between 4 and 12 mL and the final volume containing the radionuclide complex after recovering step is comprised between 13 and 24 mL.
15. The method of any one Embodiments 1-14, wherein
   (i) said radionuclide precursor solution is a ¹⁷⁷LuCl₃ solution at 74 GBq±20% in a 1-2 mL volume, typically, 1.5 mL,
   (ii) said solution comprising the somatostatin receptor binding peptide linked to a chelating agent is a solution comprising 2 mg±5% of DOTA-TATE in a volume comprised between 1.5 and 2.5 mL, typically 2 mL,
   (iii) said reaction buffer solution comprises 157 mg of gentisic acid±5% in a volume comprised between 1.5 and 2.5 mL, typically 2 mL, and the pH of the reacting step is comprised between 4.5 and 5.5.
16. The method of any one Embodiments 1-14, wherein
   (i) said radionuclide precursor solution is a ¹⁷⁷LuCl₃ at 148 GBq±20% in a 2-3 mL volume, typically, 2.5 mL,
   (ii) said solution comprising the somatostatin receptor binding peptide linked to a chelating agent is a solution comprising 4 mg±5% of DOTA-TATE in a volume comprised between 3.5 and 4.5 mL, typically 4 mL,
   (iii) said reaction buffer solution comprises 314 mg of gentisic acid±5% in a volume comprised between 3.5 and 5.5 mL, typically 4 mL, and the pH of the reacting step is comprised between 4.5 and 5.5.
17. The method of any one of Embodiments 1-16, wherein the yield of the synthesis is at least 60%.
18. The method of any one of the Embodiments 1-17, wherein the radionuclide complex recovered at step g is an aqueous concentrate mother solution comprising ¹⁷⁷Lu-DOTA-TATE at a specific activity at least equal to 45.0 GBq.
19. The method of any one of the Embodiments 1-18, wherein said radionuclide complex recovered at step g is an aqueous concentrate mother solution comprising ¹⁷⁷Lu-DOTA-TATE at a specific activity at least equal to 59.0 GBq.
20. The method of any one of Embodiments 1-19, which is automated and implemented in a synthesis module with a single use kit cassette.
21. The method of Embodiment 20, wherein said synthesis module comprises:
   a) a single use kit cassette containing the required fluid pathways, and,
   b) a single use kit containing the reagents for implementing the synthesis method.
22. The method of any one of Embodiments 1-21, wherein the synthesis takes place within a computer assisted system.
23. The method of any one of Embodiments 20-22, wherein the synthesis module and kit cassette comprises the following:
   a) at a first position, a needle is placed for inserting to the top of said first vial containing the radioactive precursor solution,
   b) at a second position, a needle is placed for inserting to the top of a vial containing said solution comprising the somatostatin receptor binding peptide linked to a chelating agent,
   c) at a third position, a bag with water for injection is installed, for rinsing steps,
   d) at a fourth position, the reaction buffer solution is installed, and,
   e) at a fifth position, an extension cable is installed to transfer the radionuclide complex from the synthesis module into a dispensing isolator.
24. The method of any one of Embodiments 1-23, further comprising the following step:
   h. diluting the radionuclide complex in a formulation buffer.
25. The method of Embodiment 24, wherein said radionuclide complex is ¹⁷⁷Lu-DOTA-TATE or ¹⁷⁷Lu-DOTA-TOC.
26. The method of Embodiment 24, wherein the formulation buffer is a solution for infusion.
27. The method of embodiment 1-26, wherein the method does not comprise any purification step to remove free (non-chelated) radionuclide, preferably, the method does not comprise a tC18 solid phase extraction (SPE) purification step.
28. An aqueous pharmaceutical solution comprising a radionuclide complex, which solution is obtainable or directly obtained by the method of any one of Embodiments 1-27.
29. The solution of Embodiment 28, which is a mother solution of ¹⁷⁷Lu-DOTA-TATE or ¹⁷⁷Lu-DOTA-TOC.
30. The solution of Embodiment 29, which is a mother solution of ¹⁷⁷Lu-DOTA-TATE or ¹⁷⁷Lu-DOTA-TOC with a specific activity concentration higher than 1875 MBq/mL, for example between 1875 and 3400 MBq/mL.
31. The solution of Embodiment 28, which is a solution for infusion of ¹⁷⁷Lu-DOTA-TATE or ¹⁷⁷Lu-DOTA-TOC.
32. The solution of Embodiment 29, which is a solution for infusion of ¹⁷⁷Lu-DOTA-TATE at 370 MBq/mL±5%.
33. A kit cassette for carrying out the method as defined in any one of the embodiments 1-27, comprising:
   a) a first vessel containing the reaction buffer solution or a lyophilisate of said buffer reaction solution,
   b) a second vessel containing a solution comprising said somatostatin receptor binding peptide linked to a chelating agent, preferably DOTA-TATE or DOTA-TOC, and,
   c) a third vessel containing said radionuclide precursor solution.

### EXAMPLES

### Example 1

### Production of a Sterile, Aqueous Concentrated Solution of ¹⁷⁷Lu-DOTA-TATE (So-Called Mother Solution)

1.1 Introduction

The radioactive Drug Substance ¹⁷⁷Lu-DOTA-TATE, also referred hereafter as 177Lu-DOTA0-Tyr³-Octreotate is produced as a sterile, aqueous concentrated solution (so-called Mother Solution).

Drug Substance synthesis steps are performed in a self-contained closed-system synthesis module which is automated and remotely controlled by GMP compliant software and automated monitoring and recording of the process parameters.

During each production run of the synthesis module, a single use disposable kit cassette, containing a fluid pathway (tubing), reactor vial and sealed reagent vials is used. The synthesis module is protected from manual interventions during the production run. The synthesis module is placed in a lead-shielded hot cell providing supply of filtered air.

The synthesis of the Drug Substance (¹⁷⁷Lu-DOTA0-Tyr³-Octreotate) and its formulation into the Drug Product (¹⁷⁷Lu-DOTA0-Tyr³-Octreotate 370 MBq/mL solution for infusion), is part of an automated continuous process which does not allow for isolation and testing of Drug Substance due to its radioactive decay.

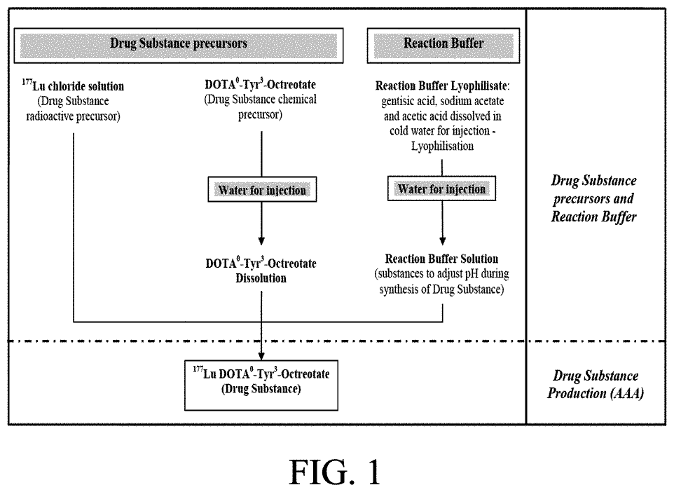

The general manufacturing process and corresponding steps are illustrated in FIGS. 1 and 2.

1.2 Preparation of Starting Materials

The chemical precursors, radioactive precursor and intermediate of drug substance used in the manufacturing process are prepared according to the following Table 1.

TABLE 1

| Component | Method of Preparation |
|---|---|
| Chemical Precursor | Solid phase synthesis purification and isolation |
| of Drug Substance | of DOTA-TATE (TFA salt) lyophilized, also |
| | called DOTA-Tyr³-Octreotate) |
| Radioactive | Neutron bombardment of enriched Lu-176 in a |
| precursor of | nuclear reactor to manufacture a Lu-177 chloride |
| Drug Substance | solution in dilute hydrochloric acid |
| Intermediate of | Reaction Buffer Lyophilisate (RBL) containing |
| Drug Substance | gentisic acid, and sodium acetate. |

The details of the reaction buffer lyophilisate are provided below in Table 2:

TABLE 2

| | Quantity | Quantity/ | |
|---|---|---|---|
| Components | (mg/vial) | batch | Function |
| Gentisic acid | 157.5 mg | 39.38 g | Radiation |
| | | | Stability |
| | | | Enhancer |
| Acetic acid | 120.2 mg | 28.76 mL | pH adjuster |
| Sodium acetate | 164.0 mg | 41.00 g | pH adjuster |
| Water for | q.s up to | up to | Solvent |
| injections | 4 mL | 1000 mL | |

1.3 Preparation of the Synthesis Module and Kit Cassette

The manufacturing process has been validated using two different Lu-177 chloride batch sizes, 74.0 GBq±20% (2 Ci±20%) or 148.0 GBq±20% (4 Ci±20%).

The synthesis is carried out using a single use disposable kit cassette installed on the front of the synthesis module which contains the fluid pathway (tubing), reactor vial and sealed reagent vials.

Table 3 summarizes the different types of equipment and material that can be used in the manufacturing process of Drug Substance according to the batch size selected.

TABLE 3
Kit cassette and synthesis module used in the
manufacturing process of Drug Substance

| Process | Synthesis module and supplier |
|---|---|
| 74 GBq batch size | TRACERlab MX (GE Medical Systems) |
| (2 Ci batch size) | MiniAIO (TRASIS) |
| 148 GBq batch size | MiniAIO (TRASIS) |
| (4 Ci batch size) | |

1.4 Kit Cassette for MiniAIO Synthesis Module

The kit cassette is ready-to-use.

1.5 Kit Cassette for TRACERlab MX Synthesis Module

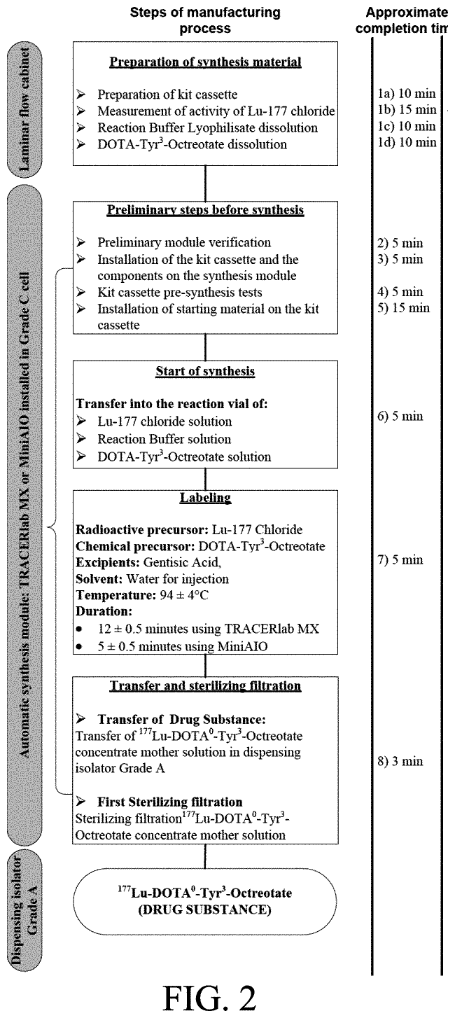

Before the start of synthesis of Drug Substance, some modifications are introduced in the kit cassette to adapt it to ¹⁷⁷Lu-DOTA⁰-Tyr³-Octreotate synthesis (see FIG. 3A and FIG. 3B corresponding to the layout of the cassette before and after modification).

The parts to be substituted are assembled under laminar flow hood (Grade A) and then installed on the synthesis module in Grade C environment.

The “Kit for Modification of the TRACERlab MX kit Cassette” consists of 2 tubes that are used to substitute 2 spikes in the original kit cassette and one connection tube to replace one cartridge and some plastic stoppers to close unused valves:
The first tube substitutes the spike in position 3 of the kit cassette,
The second tube substitutes the spike in position 5 of the kit cassette,
The connection tube (shorter) is used for replacing the first tC18 cartridge that normally connects manifold 2 with manifold 3,
Alumina cartridge and the second tC-18 cartridge are removed from position 11 and 12,
The tube previously connected from tC18 cartridge in position 12 and position 13 is connected directly in position 12 and at the other extremity to the extension cable (the prolongator used to transfer the Drug Substance into the dispensing hot cell Grade A),
Positions 9, 10, 11 and 13 are closed with plastic stoppers.

1.6 Step 1c: Reaction Buffer Lyophilisate Dissolution

Before its use in the Drug Substance synthesis, Reaction Buffer Lyophilisate (RBL) is reconstituted by Drug Substance manufacturing site by dissolution with water for injection (WFI) to obtain Reaction Buffer solution.

Reconstitution is carried out immediately before the start of the synthesis.

To dissolve the RBL:

For 74 GBq batch size (2 Ci batch size): one vial of RBL is reconstituted with 2 mL of WFI using a sterile, disposable syringe.

For 148 GBq batch size (4 Ci batch size): two vials of RBL are reconstituted with 2 mL of WFI per vial using a sterile, disposable syringe. The content of one solubilised Reaction Buffer vial is transferred into the other one using a sterile disposable syringe, and mixed up in order to obtain one vial containing 4 mL of product.

After reconstitution, the composition of Reaction Buffer is as described in Table 4.

TABLE 4
Reaction Buffer compositions after reconstitution

| | Acceptance | Reference to | |
|---|---|---|---|
| Components | Limit | standards | Function |
| Gentisic Acid | 157.5 ± 5% mg | In-house | Radiation |
| | | | Stability |
| | | | Enhancer |
| Acetic Acid | 120.2 ± 5% mg | In-house | pH adjuster |
| Sodium Acetate | 164.0 ± 5% mg | Ph.Eur. 0411/USP | pH adjuster |
| Water for | qs 2.00 mL | Ph.Eur. 0169/ USP | Solvent |
| Injection (WFI) | | | |

1.7 Step 1d: DOTA-Tyr³-Octreotate Dissolution (Chemical Precursor)

DOTA-Tyr³-Octreotate is provided as a dry powder in vial. Each vial is of 2 mg of DOTA-Tyr³-Octreotate. Before the synthesis reaction, DOTA-Tyr³-Octreotate is dissolved in water for injection (WFI).

To dissolve the DOTA-Tyr³-Octreotate:
For 74 GBq batch size (2 Ci batch size): one vial of DOTA-Tyr³-Octreotate is reconstituted with 2 mL of WFI using a sterile, disposable syringe.
For 148 GBq batch size (4 Ci batch size): two vials of DOTA-Tyr³-Octreotate are reconstituted with 2 mL of WFI per vial. The content of one solubilised DOTA-Tyr³-Octreotate vial is transferred into the other one using a sterile disposable syringe, and mixed up in order to obtain one vial containing 4 mL of product.

1.8 Step 3: Installation of the Kit Cassette and Components on the Synthesis Module

The kit cassette assembly is mounted on the front of the corresponding synthesis module. Additional components are installed on the corresponding cassette positions according to the synthesis module. The assembling is performed in a Grade C environment.
Positions used on GE Medical System modified kit cassette with TRACERlab MX synthesis module
   Position 1-left: Millex Gas filter (hydrophobic membrane), sterile, connected to the air inlet of the synthesis module,
   Position 4 and 14: Crimping of the two sterile 30 mL syringes Luer Lock¹ onto the corresponding syringe driver,
   Position 3: at the extremity of the tube, a needle is placed (this needle will be inserted to the top of the vial to draw DOTA-Tyr³-Octreotate chemical precursor),
   Position 5: at the extremity of the tube, a needle is placed, (this needle will be inserted to the top of the vial to draw ¹⁷⁷LuCl₃ solution (radioactive precursor),
   Position 12: An extension cable⁶ is connected to transfer the Drug Substance from the synthesis module into the dispensing isolator (Grade A).

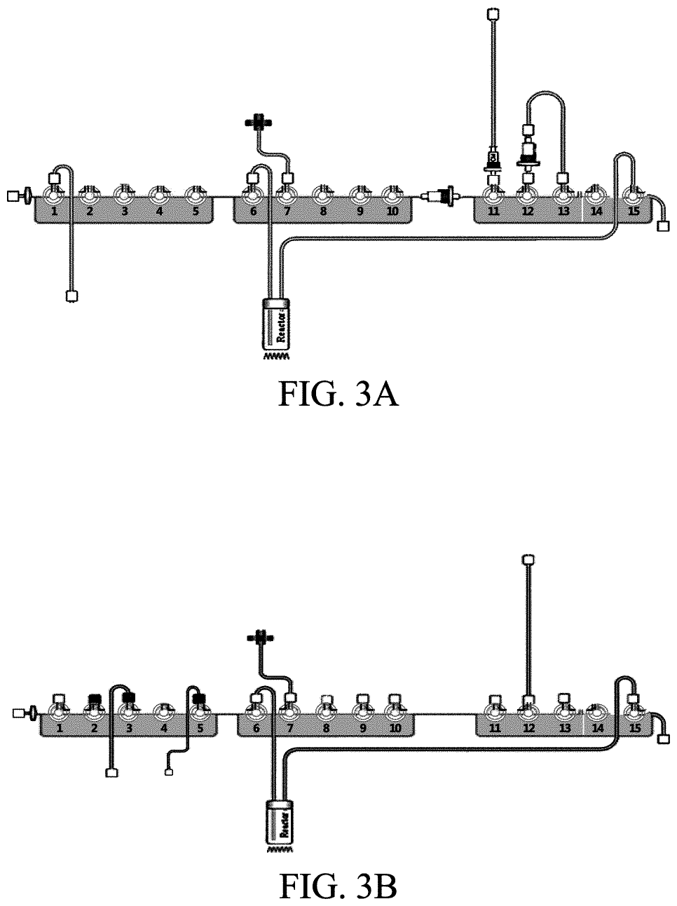

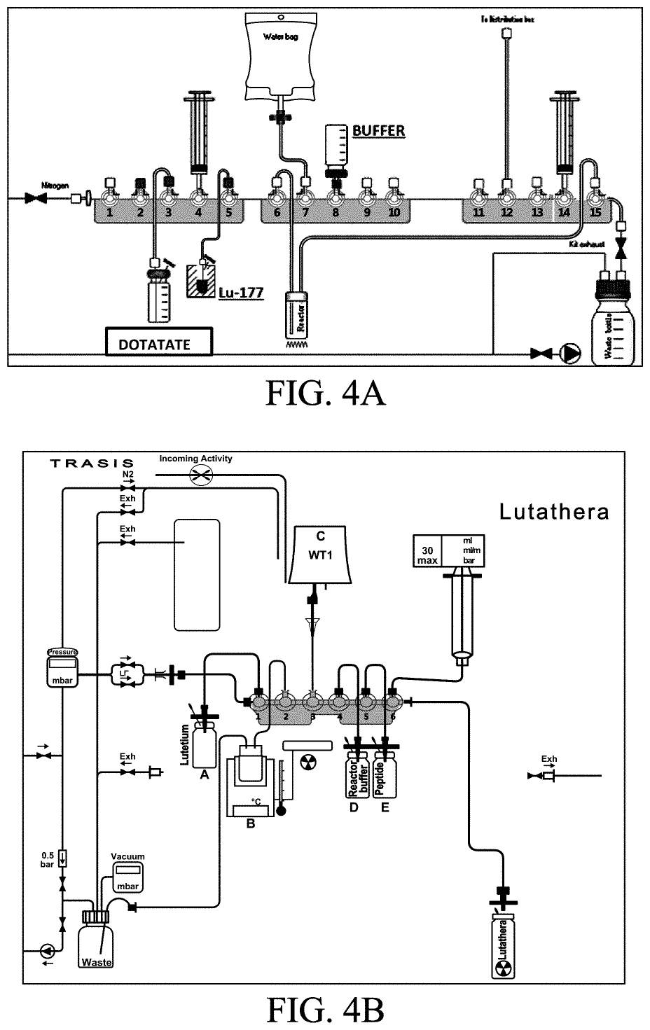

The final cassette installation is as shown in FIG. 4A.
Positions used on TRASIS kit cassette with TRASIS synthesis module
   The required components are installed at the following cassette positions:
      Position 1-up: a needle is placed (this needle will be inserted to the top of the vial to draw ¹⁷⁷LuCl₃ solution radioactive precursor),
      Position 1-left: The gas filter connected to the kit cassette in position 1-left is connected to the gas inlet,
      Position 4: a needle is placed (this needle will be inserted to the top of the vial to draw Reaction Buffer solution),
      Position 5: a needle is placed (this needle will be inserted to the top of the vial to draw DOTA-Tyr³-Octreotate chemical precursor),
      Position 6-right: Extension cable, connected to transfer the Drug Substance from the synthesis module into the dispensing isolator (Grade A),
      Position 6-up: sterile 20 mL syringe Luer Lock is connected.

The final cassette installation is as shown in FIG. 4B.

1.9 Step 5: Installation of Starting Material on the Kit Cassette

Reaction Buffer solution, WFI and precursors are installed on the corresponding cassette positions according to the synthesis module used. The installations are performed in a Grade C environment.

Positions of Synthesis Reaction Components on GE Medical System Modified Kit Cassette with TRACERlab MX Synthesis Module
Position 3: the needle is inserted to the top of the vial to draw DOTA-Tyr³-Octreotate chemical precursor. A vent filter^{s} is also inserted into the vial septum,
Position 5: the needle is inserted to the top of the vial to draw ¹⁷⁷LuCl₃ solution (radioactive precursor). A vent filter is also inserted into the vial septum,
Position 7: WFI bag is installed,
Position 8: the Reaction Buffer solution vial is installed.

The final cassette installation is as shown in FIG. 4A.

Positions of Synthesis Reaction Components on TRASIS Kit Cassette with TRASIS Synthesis Module
Position 1-up: the needle is inserted to the top of the vial to draw ¹⁷⁷LuCl₃ solution radioactive precursor. A vent filter is also inserted into the vial septum,
Position 3: WFI bag is installed,
Position 4: the needle is inserted to the top of the vial to draw Reaction Buffer solution. A vent filter is also inserted into the vial septum,
Position 5: the needle is inserted to the top of the vial to draw DOTA-Tyr³-Octreotate chemical precursor dissolved in WFI. A vent filter is also inserted into the vial septum,

The final cassette installation is as shown in FIG. 4B.

1.10 Step 6: Transfer of Lu-177 Chloride Solution, Reaction Buffer Solution and DOTA-Tyr³-Octreotate Solution into the Reactor

The synthesis is initiated by pushing the “start synthesis” button on the synthesis module PC control software program. The first step of the synthesis consists of the automated transfer of all components needed for the labeling into the cassette reactor.

Radioactive and chemical Drug Substance precursors and Reaction Buffer solution are transferred into the reactor in the following order:
1. Lu-177 chloride solution
2. Reaction Buffer solution
3. DOTA-Tyr³-Octreotate solution

The Lu-177 chloride solution is drawn into the reactor when the valves (positions 5 and 6 of the GE cassette or positions 1 and 2 of the MiniAIO cassette), are opened and negative pressure is applied to the reactor.

The Lu-177 chloride solution is highly concentrated and therefore incomplete transfer of the solution into the reactor 1 can impact the labeling yield. For this reason, the Reaction Buffer solution is added to the Lu-177 chloride solution vial before its transfer into the reactor in order to ensure complete transfer of the Lu-177 chloride solution. Reaction Buffer is transferred into Lu-177 chloride vial using syringe (right 30 mL syringe¹ for TRACERlab MX synthesis module and 30 mL syringe² for MiniAIO synthesis module). From this vial, the solution (Reaction Buffer+Lu-177 residual) is transferred into the reactor by applying negative pressure.

The last step to initiate synthesis of the Drug Substance is the transfer of the DOTA-Tyr³-Octreotate solution to the reactor. This is automatically performed by negative pressure applied to the reactor.

1.11 Step 7: Labeling Step

The synthetic route is summarized as follows:

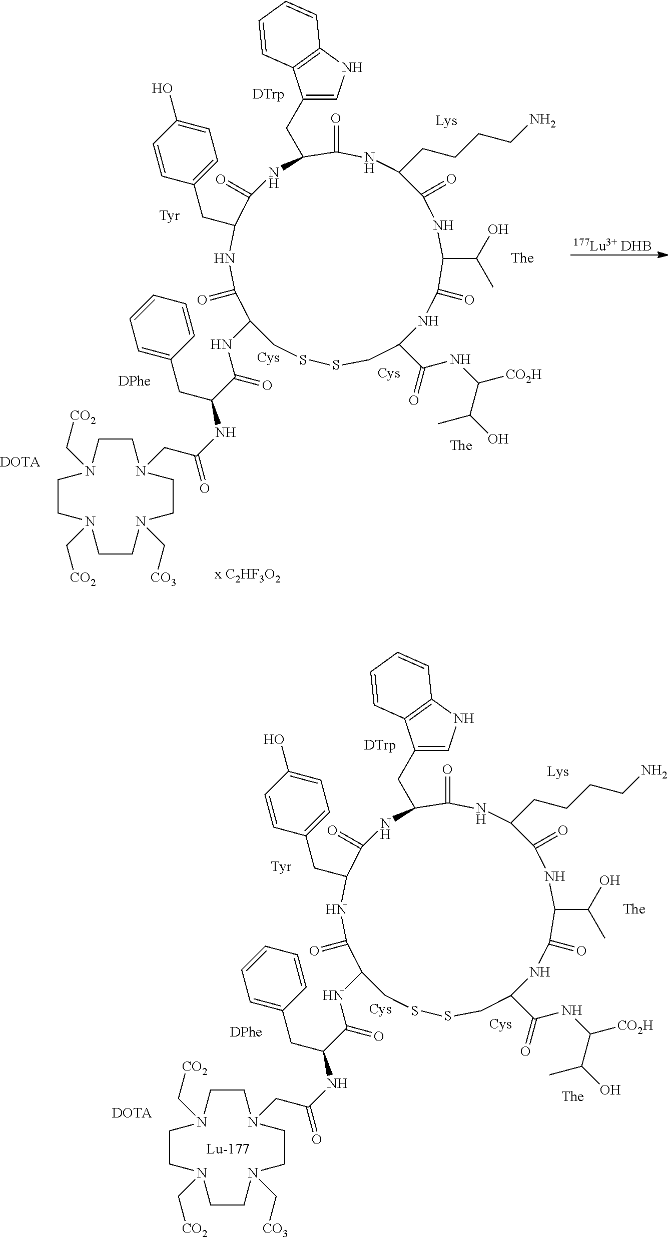

With DHB=gentisic acid (2,5-dihydroxybenzoic acid)

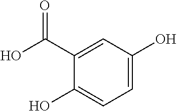 [SMILES: O=C(O)c1cc(O)ccc1O]

The labeling consists of the chelating of Lu-177 into the DOTA moiety of the DOTA-Tyr³-Octreotate peptide. The labeling is carried out at 94° C. (±4° C.) for:
12 minutes (±0.5 minutes) using TRACERlab MX (GE) synthesis module
5 minutes (±0.5 minutes) using MiniAlO (TRASIS) synthesis module

In the reactor, DOTA-Tyr³-Octreotate is present in a molar excess respect to Lu-177 to ensure acceptable radiochemical labeling yields (see also Example 2 related to the process optimization).

1.12 Step 8: Transfer and First Filtration of Drug Substance (Prefiltration)

Once the synthesis is finished in the synthesis module, ¹⁷⁷Lu-DOTA⁰-Tyr³-Octreotate Mother Solution obtained is sterilized a first time using a sterilizing filter connected to the extension sterile cable. During the filtration, the ¹⁷⁷Lu-DOTA⁰-Tyr³-Octreotate Mother Solution is automatically transferred by positive nitrogen pressure from the synthesis hot-cell (Grade C) into the dispensing isolator Grade A by the extension sterile cable and is collected in an intermediate 30 mL sterile vial. A vent filter with a microlance needle is used to equilibrate pressure in the intermediate 30 mL sterile vial.

The cassette and the reactor are rinsed 3 times with 3 mL of water for injection each time, in order to recover ¹⁷⁷Lu-DOTA⁰-Tyr³-Octreotate remaining in the lines.

The volume of ¹⁷⁷Lu-DOTA⁰-Tyr³-Octreotate Mother Solution at the end of the transferring process is:
For the 74 GBq batch size (2 Ci batch size): ≥13.0 mL
For the 148 GBq batch size (4 Ci batch size): ≥19.0 mL

The volume and the radioactivity of the ¹⁷⁷Lu-DOTA⁰-Tyr³-Octreotate Mother Solution are controlled at the end of the synthesis and monitored. The synthesis yield is calculated.

### Example 2

### Process Optimization

The process is industrialized for batch production of a larger number of doses per batch and uses an automated synthesis module for production of the Drug Substance. The process optimization considerations included:
The labeling reaction between DOTA-Tyr³-Octreotate and ¹⁷⁷Lu,
High labeling yields correlating with high radiochemical purity,
High labeling yields minimizing the level of free ¹⁷⁷Lu+3.

Starting with the process of the prior art for preparation of the Drug Substance, some changes were made to intermediate steps in particular to alter the order of addition of excipients.

In order to produce a Drug Substance formulation and to integrate the necessary excipients (i.e. one which ensures good stability of the Drug Substance solution) into the automatized synthesis procedure, we modified the formulation of Reaction Mixture, which is Reaction Buffer in the present process.

In comparison to the composition of the prior art, the Reaction Buffer does not contain peptide. Also, some components have been removed to be added only when formulating the Drug Product. Specifically, ascorbic acid is not added at the time of the labeling reaction and can be included in the Formulation Buffer. This change was made because it was found that ascorbic acid has a high likelihood of precipitating in the small reaction volume used during the labeling procedure. The Reaction Buffer also contains a low concentration of sodium acetate in order to facilitate pH buffering during the labeling reaction. Studies showed that the changes have no effect on the quality characteristics of the Drug Product while remarkably improving the automation of whole synthesis with good synthesis yield.

2.1 Optimization of Drug Substance Synthesis: The Molar Ratio of Reactants

The effect of the molar ratio of DOTA-Tyr³-Octreotate to Lu-177 on radiochemical purity of Drug Substance synthesis was investigated to optimize the labeling reaction with the aim of avoiding purification steps after labeling. Note that the ¹⁷⁷Lu solution contains ¹⁷⁷Lu, ¹⁷⁶Lu, and ¹⁷⁵Lu isotopes, therefore as ¹⁷⁷Lu decays the specific activity (SA) decreases due to the increasing abundance of the stable isotopes, ¹⁷⁶Lu, and ¹⁷⁵Lu. Therefore higher Lu-177 specific activity contains less moles of “Lu”.

For the 74 GBq batch size (2 Ci batch size), the synthesis is performed with 2 mg of DOTA-Tyr³-Octreotate and 74 GBq (2 Ci) of Lu-177 (supplied as ¹⁷⁷LuCl₃); the amount of peptide is doubled (4 mg) for the 148 GBq batch size (4 Ci batch size). Considering that DOTA-Tyr³-Octreotate has a molecular weight of 1435.6 Da and the Lu-177 radiochemical has an specific activity at time of synthesis ranging from 499.5 to 1110 GBq/mg, the molar ratio of DOTA-Tyr³-Octreotate to Lu increases from 1.5 to 3.5 (see Table 5).

Further tests show that the minimum specific activity of Lu-177 allowed at the time of synthesis is 407 GBq/mg (molar ratio of peptide:Lu=1.2) as the resulting radiochemical purity for the Drug Substance still meets specification.

TABLE 5
Molar ratio of DOTA-Tyr³-Octreotate to ¹⁷⁷Lu for Drug Substance synthesis

| | | Molecular weight | | |
|---|---|---|---|---|
| Starting | | (Da)/Specific | Mol | Molar ratio |
| material | Amount | Activity (GBq/mg) | (μmol) | (peptide:Lu) |
| DOTA-Tyr³- | 2 mg | 1435.6 Da | 1.39 | 1.5-3.5 |
| Octreotate | 4 mg | | 2.78 | |
| ¹⁷⁷Lu | 74 GBq | 499.5-1110 GBq/mg* | 0.93-0.40 | |
| | 148 GBq | | 1.86-0.80 | |

*Specific Activity values are at time of synthesis

In order to ensure efficient radiolabeling, DOTA-Tyr³-Octreotate should be present in molar excess to Lu-177. Under these conditions, no free Lu-177 is expected at the end of the synthesis; therefore no purification steps are needed at the end of the labeling.

2.2 Study of Chemical Physical Properties and Optimization of the pH

Some of the non-clinical studies were performed using a non-radioactive analogue of the Drug Substance, ¹⁷⁵Lu-DOTA⁰-Tyr³-Octreotate. The ¹⁷⁵Lu-DOTA⁰-Tyr³-Octreotate is produced using naturally occurring lutetium, 97.4% of which is composed of the isotope Lu-175. ¹⁷⁵Lu has an atomic mass of 175 Da. The non-radioactive ¹⁷⁵Lu-DOTA⁰-Tyr³-Octreotate has chemical-physical properties identical to the radioactive Drug Substance.

The production of ¹⁷⁵Lu-DOTA⁰-Tyr³-Octreotate was in compliance with the nonclinical protocol using DOTA-Tyr³-Octreotate and ¹⁷⁵Lu as starting materials. The synthesis was performed using the same synthesis module used for the production of ¹⁷⁷Lu-DOTA⁰-Tyr³-Octreotate and using the same reaction conditions (pH and reactor temperature).

Gentisic acid was omitted from the Reaction Buffer because it was not needed as a free radical scavenger.

The characterization of the cold Drug Substance included RP-HPLC for conformation identity and determination of purity and Mass Spectrometry for determination of molecular weight (identity).

It was established that the pH of the Reaction Buffer during the synthesis of the Drug Substance is an important factor to control and prevent formation of colloids. When pH is >7, Lu can transform to Lu(OH)⁻₄, a colloid form. It was found that when the pH of the Reaction Buffer is between 4.5 and 5.5 the formation of colloid is prevented and optimal labeling occurs.

2.3 Optimization of Synthesis Parameters

During the process development, critical steps have been identified in the synthesis of ¹⁷⁷Lu-DOTA⁰-Tyr³-Octreotate.

2.3.1 Labeling Yield

The labeling reaction between DOTA-Tyr³-Octreotate and ¹⁷⁷Lu is a critical step, therefore the labeling yield was determined using an in-process sample. The metal-DOTA complex formation between DOTA-Tyr³-Octreotate and Lu is a spontaneous reaction; Lu³⁺ is chelated by DOTA: oxygen electrons from the DOTA carboxy-groups are shared with the free Lu³⁺ shells.

2.3.2 Reaction Time

While the labeling reaction is spontaneous, the activation energy is high so reaction time can be very long if labeling takes place at room temperature (25° C.).

Reaction time has been optimized by determining the radiochemical purity (at the selected ratio of DOTA-Tyr³-Octreotate:Lu) at different reaction times at 95° C.

The reaction time range was validated between 2 and 15 minutes. The selected reaction time range was between 5 and 12 minutes according the different module of synthesis.

2.3.3 Reaction Temperature

The reaction temperature has been tested between 80° C. and 100° C. for labeling times of 5 minutes.

Generally, temperatures lower than 90° C. do not ensure quantitative labeling yields (a safety margin was considered); while at temperatures higher than 95° C. solution losses from solvent evaporation become an issue, and also have no impact on labeling yields. The effect of reactor temperatures of 80 and 100° C. on radiochemical purity is shown in Table 6.

TABLE 6
Effect of reaction temperature on radiochemical purity

| | Reaction | Reactor | RCP | RCP |
|---|---|---|---|---|
| | time | temperature | (%) | (%) |
| Batch number | (min) | (° C.) | t0 | t₇₂ₕ |
| LT141013B-03 | 5 | 80 | 98.7 | 95.9 |
| LT141013C-03 | 5 | 100 | 98.6 | 95.8 |

Radiochemical purity; t₀: end of synthesis; t₇₂ₕ: 72 hours from end of synthesis

The temperature range was validated between 80 and 100° C. The selected reaction temperature was fixed at 94° C. with an acceptable variation of ±4° C. (90-98° C.)

2.3.4 Reaction Volume

The reaction volume (volume of the reagent solution into the reactor) was tested for a range of activities between 37 GBq (1 Ci) and 185 GBq (5 Ci). For both batch sizes the stoichiometric ratio between reagents were kept fixed (1 μg of DOTA-Tyr³-Octreotate per 1 mCi of Lu-177). Both production processes were performed at a 5 min reaction time using MiniAIO synthesis module and at a reactor temperature of 95° C. Molar ratio of DOTA-Tyr³-Octreotate:Lu was fixed at 1.5.

Table 7 shows the effect of reaction volumes on the resulting radiochemical purity. The table shows the results of tests using reaction solutions with a radioactive concentration of 6.17 GBq/mL (181.8 mCi/mL) and 16.82 GBq/mL (454.5 mCi/mL).

TABLE 7
Effect of reaction volume on radiochemical purity at t₀

| | | Reactor | Radioactive | RCP |
|---|---|---|---|---|
| | ¹⁷⁷LuCl3 | volume | concentration | (%) |
| Batch number | (mCi) | (mL) | (mCi/mL) | t0 |
| LT131118A-03 | 1000 | 5.5 | 181.8 | 98.7 |
| LT140331A-03 | 5000 | 11.0 | 454.5 | 98.2 |

Radiochemical purity; t_{0:} end of synthesis

Reaction volume has been set to:

For 74 GBq batch size (2 Ci) production process: 5.5 mL

For 185 GBq batch size (5 Ci) production process: 11.0 mL:

2.3.5 Reaction Buffer pH

The pH of the reaction solution must be:
Below pH 7 (to prevent Lu-colloidal formation)
Higher than pH 3 (below pH 3 the DOTA-ligand is protonated and metal-complex formation is less efficient)

Drug Substance starting materials (Lu-177, DOTA-Tyr³-Octreotate and Reaction Buffer) are designed such that the pH of the reaction solution ranges between pH 4.2 and 4.7. The effect of reaction buffer pH on radiochemical purity and purity is shown in Table 8.

TABLE 8
Effect of reaction buffer pH on radiochemical purity

| | Reaction | RCP ITLC | RCP HPLC |
|---|---|---|---|
| Batch number | Buffer pH | (%) t₀ | (%) t₀ |
| LT141014B-03 | 3 | 100 | 98.9 |
| LT141014A-03 | 7 | 82 | Not performed* |
| LT141014C-03 | 4.0 | 100 | 98.8 |
| LT141014D-03 | 5.5 | 100 | 98.5 |

RCP: radiochemical purity; t0: end of synthesis
*HPLC analysis not performed to avoid potential Lu-177 colloidal injection into analytical column

From the data obtained in these tests the suitable pH range for labeling has been set between 4.0 to 5.5, while the expected reactor pH range is 4.2-4.7.

2.3.6 Reaction Buffer Lyophilisate Manufacturing Process

As part of an industrialized process it is preferable to limit the number of extemporaneously compounded materials in the process. Therefore the Reaction Buffer solution was designed to be reconstituted from a lyophilisate vial rather than from starting components.

1-25. (canceled)

26. A pharmaceutical formulation comprising ¹⁷⁷Lu-DOTA-TATE obtained by a method comprising the steps of:
a) providing a ¹⁷⁷LuCl₃ solution into a first vial,
b) transferring the ¹⁷⁷LuCl₃ solution into a reactor,
c) providing a reaction buffer solution into the first vial containing residual radionuclide precursor solution,
d) transferring the reaction buffer solution and residual ¹⁷⁷LuCl₃ solution from said first vial into the reactor,
e) transferring a DOTA-TATE solution into the reactor,
f) reacting the DOTA-TATE with the ¹⁷⁷LuCl₃ in the reactor to obtain the ¹⁷⁷Lu-DOTA-TATE,
g) recovering the ¹⁷⁷Lu-DOTA-TATE, and
h) diluting the ¹⁷⁷Lu-DOTA-TATE in a formulation buffer to form a pharmaceutical formulation, wherein the formulation buffer comprises at least one sequestering agent, at least one radiolytic stabilizer, at least one pH adjuster, at least one solvent, and sodium chloride; and
wherein the pharmaceutical formulation has a pH of about 4.5 to about 6, and wherein the ¹⁷⁷Lu-DOTA-TATE in the pharmaceutical formulation has a specific activity concentration of 370 MBq/mL.

27. The pharmaceutical formulation of claim 26, wherein the at least one sequestering agent is diethylene triamine pentaacetic acid; the at least one radiolytic stabilizer is gentisic acid and ascorbic acid; the at least one pH adjuster is sodium hydroxide, acetic acid, and sodium acetate; and
the at least one solvent is water.

28. The pharmaceutical formulation of claim 27, wherein the diethylene triamine pentaacetic acid is present at a concentration of 0.05 mg/mL, the gentisic acid is present at a concentration of 0.63 mg/mL, the ascorbic acid is present at a concentration of 2.8 mg/mL, the sodium hydroxide is present at a concentration of 0.65 mg/mL, the acetic acid is present at a concentration of 0.48 mg/mL, the sodium acetate is present at a concentration of 0.66 mg/mL, and the sodium chloride is present at a concentration of 6.85 mg/mL.

29. The pharmaceutical formulation of claim 26, wherein the pharmaceutical formulation is for an infusion to treat a subject in need thereof.

30. The pharmaceutical formulation of claim 26, wherein the reacting step f) occurs over a time period of between 2 and 15 minutes at a temperature of between 80-100° C.

31. The pharmaceutical formulation of claim 26, wherein the method to obtain the pharmaceutical formulation further comprises one or more rinsing steps for efficient recovery of the ¹⁷⁷Lu-DOTA-TATE.

32. The pharmaceutical formulation of claim 26, wherein the ¹⁷⁷LuCl3 solution comprises ¹⁷⁷LuCl3 at a specific activity of 74 GBq±20% in a volume of between 1 and 2 mL.

33. The pharmaceutical formulation of claim 32, wherein the DOTA-TATE solution comprises 2 mg±5% of DOTA-TATE in a volume of between 1.5 and 2.5 mL.

34. The pharmaceutical formulation of claim 33, wherein the reaction buffer solution comprises 157 mg of gentisic acid±5% in a volume of between 1.5 and 2.5 mL.

35. The pharmaceutical formulation of claim 34, wherein the pH of the reacting step f) is between 4.5 and 5.5.

36. The pharmaceutical formulation of claim 26, wherein the ¹⁷⁷Lu-DOTA-TATE has a specific activity at the reacting step f) of at least 407 GBq/mg.

37. A pharmaceutical formulation comprising ¹⁷⁷Lu-DOTA-TATE obtained by a method comprising the steps of:
a) providing a ¹⁷⁷LuCl₃ solution at 74 GBq±20% into a first vial,
b) transferring the ¹⁷⁷LuCl₃ solution into a reactor,
c) providing a reaction buffer solution comprising gentisic acid into the first vial containing residual radionuclide precursor solution,
d) transferring the reaction buffer solution and the residual ¹⁷⁷LuCl₃ solution from the first vial into the reactor,
e) transferring a solution comprising 2 mg±5% of DOTA-TATE into the reactor,
f) reacting the DOTA-TATE with the ¹⁷⁷LuCl₃ in the reactor to obtain ¹⁷⁷Lu-DOTA-TATE,
g) recovering ¹⁷⁷Lu-DOTA-TATE, and
h) diluting the ¹⁷⁷Lu-DOTA-TATE in a formulation buffer, wherein the formulation buffer comprises at least one sequestering agent, at least one radiolytic stabilizer, at least one pH adjuster, at least one solvent, and sodium chloride.

38. The pharmaceutical formulation of claim 37, wherein the ¹⁷⁷LuCl3 solution comprises a volume of between about 1 to about 2 mL.

39. The pharmaceutical formulation of claim 37, wherein the DOTA-TATE solution comprises a volume of between 1.5 and 2.5 mL.

40. The pharmaceutical formulation of claim 37, wherein the reaction buffer solution comprises 157 mg of gentisic acid.

41. The pharmaceutical formulation of claim 37, wherein the ¹⁷⁷Lu-DOTA-TATE recovered at step g) has a specific activity of at least 45.0 GBq.

42. The pharmaceutical formulation of claim 37, wherein the ¹⁷⁷Lu-DOTA-TATE is diluted to a specific activity concentration of 370 MBq/mL.

43. A pharmaceutical formulation comprising ¹⁷⁷Lu-DOTA-TATE obtained by a method comprising the steps of:
a) providing a ¹⁷⁷LuCl₃ solution at 148 GBq±20% into a first vial,
b) transferring the ¹⁷⁷LuCl₃ solution into a reactor,
c) providing a reaction buffer solution comprising gentisic acid into the first vial containing residual radionuclide precursor solution,
d) transferring the reaction buffer solution and the residual ¹⁷⁷LuCl₃ solution from the first vial into the reactor,
e) transferring a solution comprising 4 mg±5% of DOTA-TATE into the reactor,
f) reacting the DOTA-TATE with the ¹⁷⁷LuCl₃ in the reactor to obtain ¹⁷⁷Lu-DOTA-TATE,
g) recovering ¹⁷⁷Lu-DOTA-TATE, and
h) diluting the ¹⁷⁷Lu-DOTA-TATE in a formulation buffer, wherein the formulation buffer comprises at least one sequestering agent, at least one radiolytic stabilizer, at least one pH adjuster, at least one solvent, and sodium chloride.

44. The pharmaceutical formulation of claim 43, wherein the ¹⁷⁷LuCl3 solution comprises a volume of between about 2 to about 3 mL.

45. The pharmaceutical formulation of claim 43, wherein the DOTA-TATE solution comprises a volume of between 3.5 and 4.5 mL.

46. The pharmaceutical formulation of claim 43, wherein the reaction buffer solution comprises 314 mg of gentisic acid.

47. The pharmaceutical formulation of claim 43, wherein the ¹⁷⁷Lu-DOTA-TATE recovered at step g) has a specific activity of at least 59.0 GBq.

48. The pharmaceutical formulation of claim 43, wherein the ¹⁷⁷Lu-DOTA-TATE is diluted to a specific activity concentration of 370 MBq/mL.
